(19) United States
(12) Patent Application Publication
(10) Pub. No.: US 2021/0330566 A1
(43) Pub. Date: Oct. 28, 2021

(54) WATER-SOLUBILIZED MIXTURE OF GINSENOSIDE COMPOUND K AND METHOD OF WATERSOLUBILIZING GINSENOSIDE COMPOUND K

(71) Applicants: VENN SKINCARE, INC., Los Angeles, CA (US); VENN SKINCARE KOREA LLC, Seoul (KR)

(72) Inventor: Jae-Choel MUN, Cheongju-si, Chungcheongbuk-do (KR)

(21) Appl. No.: 16/483,071

(22) PCT Filed: Feb. 12, 2018
(86) PCT No.: PCT/KR2018/001812

§ 371 Date: Aug. 2, 2019

(51) Int. Cl. A61K 8/60; A61K 8/34; A61Q 19/00

(52) U.S. Cl. CPC A61K 8/60; A61K 8/34; A61K 2800/805; A61K 2800/48; A61K 2800/49; A61Q 19/00

(57) ABSTRACT

Disclosed is a water-solubilized mixture of ginsenoside compound K manufactured by mixing hexanediol and polyol with ginsenoside compound K and then adding water, and a method of water-solubilizing ginsenoside compound K. The method includes manufacturing a mixture of ginsenoside compound K powder and hexanediol by adding hexanediol to ginsenoside compound K powder and performing mixing, manufacturing a mixture of ginsenoside compound K powder, hexanediol, and polyol by adding polyol to the mixture of ginsenoside compound K powder and hexanediol and performing mixing, and manufacturing an aqueous solution containing ginsenoside compound K by adding the mixture of ginsenoside compound K powder, hexanediol, and polyol to water at 65 to 75° C. during agitation and performing mixing. The water solubility of ginsenoside compound K is improved, and cosmetics including high concentrations of ginsenoside compound K are readily manufactured.

### TECHNICAL FIELD

The present invention relates to a water-solubilized mixture of ginsenoside compound K and a method of water-solubilizing ginsenoside compound K. Specifically, the present invention relates to a water-solubilized mixture of ginsenoside compound K, the water solubility of which is improved by mixing hexanediol and polyol, and a method of water-solubilizing ginsenoside compound K.

### BACKGROUND ART

Ginsenoside compound K (20-O-β-D-glucopyranosyl-20(S)-protopanaxadiol) has been actively studied as a compound having various effective functions, such as immuno-enhancing activity, inhibition of tumor angiogenesis, and inhibition of cancer cell infiltration, and various skin-protection functions, such as antioxidant effects and anti-aging functions, through various efficacy experiments, in the field of medical supply, health supplement, and cosmetics. Products, such as medical supplies, health supplements, and cosmetic products, using ginsenoside compound K have been actively developed.

Ginsenoside compound K is not one of the elements of ginsenoside of ginseng, but it is obtained by mixing one or more of various elements of ginsenoside, that is, Rb1, Rb2, Rc, and Rd, that are contained in large amounts in ginseng, via various processes. However, because the amount of ginsenoside compound K that can be obtained from ginseng is small, methods for more stably producing ginsenoside compound K in larger amounts are being researched and developed. However, there is lack of research on which solvent can be used to dissolve ginsenoside compound K and how ginsenoside compound K can be used in products.

Skin is the primary protective membrane of the human body, protects the internal organs of the human body from changes in temperature and humidity and from external environmental stimuli such as ultraviolet rays and pollutants, and plays an important role in maintaining homeostasis such as body temperature control. However, excessive physical and chemical stimuli, which are externally received, stress, and nutritional deficiencies reduce the normal functions of skin and promote skin aging, including loss of elasticity, keratinization, and wrinkle formation. In order to prevent this phenomenon and to maintain healthier and more resilient skin, conventional efforts have been conducted to maintain the skin's natural functions and to activate skin cells, thus effectively inhibiting skin aging, using cosmetics including enhanced bioactive substances obtained from various animals, plants, and microorganisms. Researches into raw materials having anti-aging effects on skin without inducing skin side-effects are being actively carried out. Among these researches, interest in ginsenoside compound K is very high and continual research of ginsenoside compound K is underway. However, there still remains a matter of how ginsenoside compound K, which is a very poorly soluble substance, can be dissolved in a solvent to be used in manufacturing cosmetics.

Ginsenoside compound K is a substance obtained by a bio-conversion technology using microorganism fermentation from ginseng saponin, which is the main bioactive component of ginseng. Ginsenoside compound K that is obtained through such bioconversion process is known to have various medical functions in the human body, and is also known to have collagen synthesis, acne symptom improvement, antioxidant function, and anti-aging effects on human skin. However, ginsenoside compound K is not soluble in water, because it has a dammarane skeleton of the triterpenoid series. Ginsenoside compound K is also not soluble in various types of vegetable oil or ester oil. Ginsenoside compound K has partial solubility in solvents such as pyridine, ethanol, and methanol, but when a high content of ginsenoside compound K is dissolved using such solvents, precipitation occurs over time to cause settling of ginsenoside compound K and such solvents are harmful to skin and human body. Accordingly, ginsenoside compound K is difficult to use in cosmetics.

That is, since ginsenoside compound K is hydrophobic, it has very low solubility in water, making it difficult to commercialize ginsenoside compound K as beverages or cosmetics having an aqueous-solution property. An example of a conventional technology for solving the poor solubility of ginsenoside compound K is Korean Patent Laid-Open Publication No. 10-2017-0067303 (Composition of ginsenoside compound K having improved water solubility). However, this technology has the following two problems: (1) it is not environmentally friendly because an organic solvent such as cyclodextrin is used to solubilize ginsenoside compound K, and (2) it has limitations in producing solution with high concentrations of ginsenoside compound K because it solubilizes ginsenoside compound K by collecting ginsenoside compound K molecules within the cylindrical molecular structure of cyclodextrin. Therefore, there remains a need to develop new technology for water-solubilizing ginsenoside compound K in order to effectively apply ginsenoside compound K to various types of food, beverages, and cosmetics, and thereby deliver ginsenoside compound K's various useful effects on human body and skin including antioxidant effects and anti-aging functions.

### PRIOR ART DOCUMENT

### Patent Document

(Patent Document 0001) Korean Patent Laid-Open Publication No. 10-2017-0067303 (Composition of ginsenoside compound K having improved water solubility)

### DISCLOSURE OF INVENTION

### Technical Problem

Accordingly, the present invention has been made, addressing the aforementioned problems of the prior art, and the objective of the present invention is to create a water-solubilized mixture of ginsenoside compound K with improved water solubility and a method of water-solubilizing ginsenoside compound K. The water solubility of ginsenoside compound K, which is a very poorly soluble substance, is improved, and a solution state is stably maintained over a long period of time.

Another objective of the present invention is to manufacture a variety of products such as cosmetics using a water-solubilized mixture of ginsenoside compound K at relatively high concentrations.

A further objective of the present invention is to provide a stable water-solubilized mixture of ginsenoside compound K having constancy and a method of water-solubilizing ginsenoside compound K. The water solubility thereof is not affected by surroundings such as temperature or humidity, and ginsenoside compound K is not precipitated from the water-solubilized mixture of ginsenoside compound K at high concentrations over time.

Another objective of the present invention is to manufacture various types of foods, beverages, and cosmetics using a water-solubilized mixture of ginsenoside compound K having improved water solubility so that such products have relatively high concentrations of ginsenoside compound K, and a method of water-solubilizing ginsenoside compound K.

### Solution to Problem

In order to accomplish the above objectives, the present invention provides a water-solubilized mixture of ginsenoside compound K manufactured by mixing hexanediol and polyol with ginsenoside compound K and then adding water.

In order to accomplish the above objectives, the present invention also provides a method of water-solubilizing ginsenoside compound K. The method includes (1) manufacturing a mixture of ginsenoside compound K powder and hexanediol by adding hexanediol to ginsenoside compound K powder and performing mixing, (2) manufacturing a mixture of ginsenoside compound K powder, hexanediol, and polyol by adding polyol to the mixture of ginsenoside compound K powder and hexanediol and performing mixing, and (3) manufacturing an aqueous solution containing ginsenoside compound K by adding the mixture of ginsenoside compound K powder, hexanediol, and polyol to water at 65 to 75° C. while agitating the same and performing mixing.

### Advantageous Effects of Invention

The water-solubilized mixture of ginsenoside compound K and the method of water-solubilizing ginsenoside compound K which are the present invention have the following effects.

First, the water-solubilized mixture of ginsenoside compound K and the method of water-solubilizing ginsenoside compound K which are the present invention have an effect whereby the water solubility of ginsenoside compound K that is a poorly soluble substance is improved and a solution state is stably maintained over a long period of time, thus providing the water-solubilized mixture of ginsenoside compound K having improved water solubility and the method of water-solubilizing ginsenoside compound K.

Second, the water-solubilized mixture of ginsenoside compound K and the method of water-solubilizing ginsenoside compound K which are the present invention have an effect whereby, since the water-solubilized mixture of ginsenoside compound K is capable of being manufactured at relatively high concentrations, a variety of products such as cosmetics including high concentrations of ginsenoside compound K are readily manufactured using the water-solubilized mixture of ginsenoside compound K.

Third, the water-solubilized mixture of ginsenoside compound K and the method of water-solubilizing ginsenoside compound K which are the present invention have an effect in which the water solubility is not affected by the surroundings, such as temperatures or humidity, and in which ginsenoside compound K is not precipitated from the water-solubilized mixture of ginsenoside compound K at high concentrations over time, thus enabling the manufacture of a stable water-solubilized mixture of ginsenoside compound K having constancy.

Fourth, when using the water-solubilized mixture of ginsenoside compound K and the method of water-solubilizing ginsenoside compound K which are the present invention, it is possible to develop functional cosmetics having excellent functions and effects such as anti-oxidation, wrinkle reduction, skin whitening, anti-aging, antibacterial action, and skin protection in the field of skin, lotions, creams, essences, cleansing products, ointments, ultraviolet-ray-blocking products, and makeup.

### BRIEF DESCRIPTION OF DRAWINGS

FIG. 1 is a flowchart showing a method of water-solubilizing ginsenoside compound K which is the present invention;

FIG. 2 is a view showing HPLC quantitative analysis results of an aqueous solution of ginsenoside compound K powder (purity 32.77%) at a concentration of 10%;

FIG. 3 is a view showing HPLC quantitative analysis results of an aqueous solution of ginsenoside compound K powder (purity 32.77%) at a concentration of 20%;

FIG. 4 is a view showing HPLC quantitative analysis results of an aqueous solution of ginsenoside compound K powder (purity 32.77%) at a concentration of 30%; and

FIG. 5 is a view showing HPLC quantitative analysis results of an aqueous solution of ginsenoside compound K powder (purity 32.77%) at a concentration of 40%.

### MODE FOR THE INVENTION

It is to be understood that the terms or words used in the present specification and claims are not to be construed in a conventional or dictionary sense and that the inventor can properly define the concept of a term to describe their invention in the best possible way. Accordingly, the present invention should be construed as having a meaning and concept consistent with the technical idea of the present invention. Therefore, the embodiments described in the specification of the present invention and the constitutions shown in the drawings are merely the most preferred embodiments of the present invention, and do not represent the entire technical scope of the present invention. It should be understood that various equivalents and modifications that may be substituted for these at the time of filing of the present invention are possible or may be present.

Before describing the present invention with reference to the following Examples, it should be noted that what is not necessary in order to disclose the gist of the present invention, that is, a known constitution that can be obviously added by a person skilled in the art, is not shown in the drawings and not specifically described.

In order to use ginsenoside compound K as the raw material of cosmetics, the following conditions must be met.

i) Ginsenoside compound K must be readily soluble in water or oil.

ii) After dissolution, the dissolved ginsenoside compound K must not be precipitated over time and must not be precipitated even with changes in temperature.

iii) The dissolved ginsenoside compound K must not be reactive with the other raw cosmetic materials, but must be compatible therewith.

iv) The properties and activity of ginsenoside compound K must be maintained when manufacturing cosmetics using the dissolved ginsenoside compound K.

### COMPARATIVE EXAMPLE

The solubility of ginsenoside compound K was tested to be checked for representative polar and nonpolar solvents mainly used in cosmetics.

TABLE 1
Results of solubility tests of ginsenoside compound
K depending on type of solvent (Solubility test: 95%
of solvent and 5% of the ginsenoside compound K)

| | Solubility | | |
|---|---|---|---|
| | (room tem- | Solubility | |
| Solvent | perature) | (60° C.) | Note |
| Water | Insoluble | Insoluble | |
| Vegetable Oil | Insoluble | Insoluble | |
| Polar Ester Oil | Insoluble | Insoluble | |
| Non-Polar Ester | Insoluble | Insoluble | |
| Oil | | | |
| Mineral Oil | Insoluble | Insoluble | |
| Silicone | Insoluble | Insoluble | |
| Ethanol | Soluble | Soluble | Ginsenoside compound K |
| | | | is dissolved, but is pre- |
| | | | cipitated after a short |
| | | | period of time |
| Surfactant | Insoluble | Insoluble | |
| Glycerin | Insoluble | Insoluble | |

※ Ginsenoside compound K is dissolved in the ethanol solvent, but is precipitated after a predetermined period of time when the concentration thereof is high (5%).

The water-solubilized mixture of ginsenoside compound K and the method of water-solubilizing ginsenoside compound K which are the present invention have the following characteristics.

1) Preparation of Ginsenoside Compound K Powder

As ginsenoside compound K powder used as a bioactive substance in the present invention, ginsenoside compound K powder having a purity of 32.77% was prepared by processing Korean white fine root ginseng. However, the purity of ginsenoside compound K powder is not limited to 32.77%, and ginsenoside compound K powder may be used at various purities in the present invention.

2) Selection of constitutional components required to dissolve ginsenoside compound K powder in water to thus form water-solubilized mixture of ginsenoside compound K, and roles thereof

i) Hexanediol acts as a solvent, and among various hexanediol isomers, 1,2-hexanediol was found to have the best efficiency as the solvent.

ii) Water acts as a solvent together with hexanediol.

iii) Polyol acts as a solution stabilizer. Among various polyols, butanediol had excellent efficiency as the solution stabilizer and, among butanediol isomers, 1,3-butanediol was found to have the best efficiency as the solution stabilizer.

iv) Ethanol acts as a dispersion agent and may be selectively added in order to reduce the water-solubilization time of the ginsenoside compound K. In this case, before a step of manufacturing the mixture of ginsenoside compound K powder and hexanediol by adding hexanediol to ginsenoside compound K powder and performing mixing, ginsenoside compound K powder was mixed with ethanol and then dispersed to thus manufacture the dispersion substance of ginsenoside compound K powder, and subsequently, hexanediol was added to the dispersion substance of ginsenoside compound K powder and then mixed to thus manufacture the mixture of ginsenoside compound K powder and hexanediol.

3) The range of the composition ratio in the water-solubilized mixture of ginsenoside compound K and the method of water-solubilizing ginsenoside compound K which are the present invention is described in the following Table 2. According to Table 2, various polyols (butanediol, dipropylene glycol, glycerin, propanediol) may be used as the solution stabilizer. Polyol refers to polyhydric alcohol and also refers to an aliphatic compound having two or more hydroxyl groups (—OH).

TABLE 2

| Constitution | Range of composition ratio | | Note |
|---|---|---|---|
| Ginsenoside | 0.01~40.00 | wt % | Bioactive |
| compound K powder | | | substance |
| 1,2-hexanediol | 0.01~35.00 | wt % | Solvent |
| Water | 0.01~40.00 | wt % | Solvent |
| 1,3-butanediol | 0.01~30.00 | wt % | Solution |
| | | | stabilizer |
| Ethanol | 0.01~4.00 | wt % | Dispersion |
| | | | agent |
| Ginsenoside | 0.01~40.00 | wt % | Bioactive |
| compound K powder | | | substance |
| 1,2-hexanediol | 0.01~35.00 | wt % | Solvent |
| Water | 0.01~40.00 | wt % | Solvent |
| Dipropylene glycol | 0.01~30.00 | wt % | Solution |
| | | | stabilizer |
| Ethanol | 0.01~4.00 | wt % | Dispersion |
| | | | agent |
| Ginsenoside | 0.01~40.00 | wt % | Bioactive |
| compound K powder | | | substance |
| 1,2-hexanediol | 0.01~35.00 | wt % | Solvent |
| Water | 0.01~40.00 | wt % | Solvent |
| Glycerin | 0.01~30.00 | wt % | Solution |
| | | | stabilizer |
| Ethanol | 0.01~4.00 | wt % | Dispersion |
| | | | Agent |
| Ginsenoside | 0.01~40.00 | wt % | Bioactive |
| compound K powder | | | substance |
| 1,2-hexanediol | 0.01~35.00 | wt % | Solvent |
| Water | 0.01~40.00 | wt % | Solvent |
| 1,3-propanediol | 0.01~30.00 | wt % | Solution |
| | | | stabilizer |
| Ethanol | 0.01~4.00 | wt % | Dispersion |
| | | | agent |

In the present invention, when the composition ratio is out of the range of composition ratio in the water-solubilized mixture of ginsenoside compound K and the method of water-solubilizing the ginsenoside compound K, a problem may occur with respect to the water-solubilization of the ginsenoside compound K, which is a poorly soluble substance, and it may be difficult to manufacture the water-solubilized mixture of ginsenoside compound K at high concentrations, which makes it difficult to manufacture various products such as cosmetics including ginsenoside compound K at high concentrations. Further, when a composition ratio is out of the range of the composition ratio in the water-solubilized mixture of ginsenoside compound K and the method of water-solubilizing the ginsenoside compound K, it is difficult to manufacture the stable water-solubilized mixture of ginsenoside compound K having constancy from which ginsenoside compound K is not precipitated when the concentration of the water-solubilized mixture of ginsenoside compound K is high. Accordingly, a problem may occur in the development of functional cosmetics having desirable functions and effects such as anti-oxidation, wrinkle reduction, skin whitening, anti-aging, antibacterial action, and skin protection.

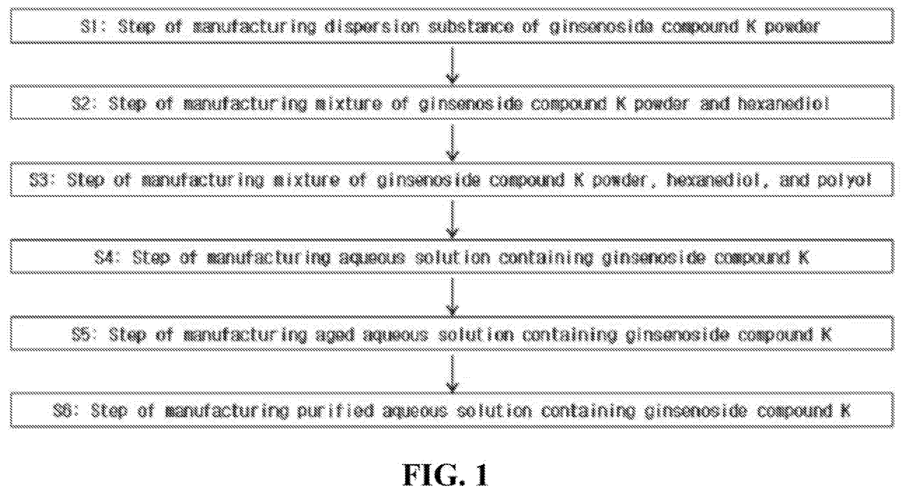

FIG. 1 is a flowchart showing the method of water-solubilizing ginsenoside compound K which is the present invention.

According to FIG. 1, the method of water-solubilizing ginsenoside compound K which is the present invention includes, in order to reduce the water-solubilization time of the ginsenoside compound K, a step of manufacturing a dispersion substance of ginsenoside compound K powder by mixing ginsenoside compound K powder with ethanol and performing dispersion (S1), a step of manufacturing a mixture of ginsenoside compound K powder and hexanediol by adding hexanediol to the dispersion substance of ginsenoside compound K powder and performing mixing (S2), a step of manufacturing a mixture of ginsenoside compound K powder, hexanediol, and polyol by adding polyol to the mixture of ginsenoside compound K powder and hexanediol and performing mixing (S3), a step of manufacturing an aqueous solution containing ginsenoside compound K by adding the mixture of ginsenoside compound K powder, hexanediol, and polyol to water at 65 to 75° C. during agitation and performing mixing (S4), a step of manufacturing an aged aqueous solution containing ginsenoside compound K by cooling the aqueous solution containing ginsenoside compound K to 30 to 40° C., followed by aging at room temperature for 2 to 4 days (S5), and a step of manufacturing a purified aqueous solution containing ginsenoside compound K by filtering the aged aqueous solution containing ginsenoside compound K (S6). In this case, in the step of manufacturing the mixture of ginsenoside compound K powder and hexanediol (S2), it is preferable to manufacture the mixture by adding 1,2-hexanediol at 55 to 65° C. to the dispersion substance of ginsenoside compound K powder and performing mixing. In the step of manufacturing the mixture of ginsenoside compound K powder, hexanediol, and polyol, it is preferable to manufacture the mixture by adding 1,3-butanediol at 55 to 65° C. to the mixture of ginsenoside compound K powder and 1,2-hexanediol and performing mixing.

When the method of water-solubilizing ginsenoside compound K which is the present invention deviates from the above-described process conditions, a problem may occur in water-solubilization of the ginsenoside compound K, which is a poorly soluble substance, and it may be difficult to manufacture the water-solubilized mixture of ginsenoside compound K at high concentrations, which makes it difficult to manufacture various products such as cosmetics including ginsenoside compound K at high concentrations.

### Example 1

An example of the method of water-solubilizing ginsenoside compound K has the following constitution.

1) Ginsenoside compound K powder having a purity of 32.77% is mixed with ethanol having a purity of 95.5% at a content ratio of 10:1, followed by dispersion for 3 minutes using a dispersing mixer, thus manufacturing a dispersion substance of the compound K powder.

2) In order to increase the solubility and reduce the dissolution time of ginsenoside compound K powder, 1,2-hexanediol, having a purity of 99.5% or more, is heated to 60° C., and heated 1,2-hexanediol is added to the dispersion substance of ginsenoside compound K powder and is dissolved using the dispersing mixer, thus manufacturing a mixture of ginsenoside compound K powder and hexanediol.

3) 1,3-butanediol, having a purity of 99.5%, is heated to 60° C., and 1,3-butanediol, which is heated, is added to the mixture of ginsenoside compound K powder and hexanediol and is mixed therewith, thus manufacturing a mixture of ginsenoside compound K powder, hexanediol, and butanediol.

4) Purified water is heated to 70° C., and the purified water that is heated is slowly added to the mixture of ginsenoside compound K powder, hexanediol, and butanediol during agitation and is mixed, thus manufacturing an aqueous solution containing the ginsenoside compound K.

5) The manufactured aqueous solution containing ginsenoside compound K is cooled to 35° C. and then aged at room temperature for 3 days, thus manufacturing an aged aqueous solution containing the ginsenoside compound K.

The manufactured aqueous solution of ginsenoside compound K is cooled to 35° C. and then aged at room temperature for 3 days.

6) The aged aqueous solution containing ginsenoside compound K is filtered using a 300-mesh filter, thus manufacturing a purified aqueous solution containing the ginsenoside compound K.
However, the purity of ginsenoside compound K powder is not limited to 32.77%, and ginsenoside compound K powder may be used at various purities. Preferably, it is possible to perform the method of water-solubilizing ginsenoside compound K using ginsenoside compound K powder having a purity of 0.1% to 98.0%.

### Experimental Example 1

The constitution and the composition ratio of a water-solubilized mixture of ginsenoside compound K at a concentration of 10% (based on the content of ginsenoside compound K powder) (Sample 1), which is manufactured using ginsenoside compound K powder having a purity of 32.77%, are described in Table 3.

TABLE 3

| Constitution | Composition ratio (wt %) | Function (role) |
|---|---|---|
| Ginsenoside compound | 10.00 | Bioactive substance |
| K powder | | |
| 1,2-hexanediol | 30.00 | Solvent |
| Purified water | 29.00 | Solvent |
| 1,3-butanediol | 30.00 | Solution stabilizer |
| Ethanol | 1.00 | Dispersion agent |
| Total | 100.00 | |

The water-solubilized mixture of ginsenoside compound K (Sample 1) that was manufactured at the above-described composition ratio had a clear yellowish brown liquid phase, and the appearance of the remaining ginsenoside compound K powder was not observed.

### Experimental Example 2

The constitution and the composition ratio of a water-solubilized mixture of ginsenoside compound K at a concentration of 20% (based on the content of ginsenoside compound K powder) (Sample 2), which is manufactured using ginsenoside compound K powder having a purity of 32.77%, are described in Table 4.

TABLE 4

| Constitution | Composition ratio (wt %) | Function (role) |
|---|---|---|
| Ginsenoside compound | 20.00 | Bioactive substance |
| K powder | | |
| 1,2-hexanediol | 30.00 | Solvent |
| Purified water | 18.00 | Solvent |
| 1,3-butanediol | 30.00 | Solution stabilizer |
| Ethanol | 2.00 | Dispersion agent |
| Total | 100.00 | |

The water-solubilized mixture of ginsenoside compound K (Sample 2) that was manufactured at the above-described composition ratio had a clear yellowish brown liquid phase, and the appearance of the remaining ginsenoside compound K powder was not observed.

### Experimental Example 3

The constitution and the composition ratio of a water-solubilized mixture of ginsenoside compound K at a concentration of 30% (based on the content of ginsenoside compound K powder) (Sample 3), which is manufactured using ginsenoside compound K powder having a purity of 32.77%, are described in Table 5.

TABLE 5

| Constitution | Composition ratio (wt %) | Function (role) |
|---|---|---|
| Ginsenoside compound | 30.00 | Bioactive substance |
| K powder | | |
| 1,2-hexanediol | 30.00 | Solvent |
| Purified water | 17.00 | Solvent |
| 1,3-butanediol | 20.00 | Solution stabilizer |
| Ethanol | 3.00 | Dispersion agent |
| Total | 100.00 | |

The water-solubilized mixture of ginsenoside compound K (Sample 3) that was manufactured at the above-described composition ratio had a clear yellowish brown liquid phase, and the appearance of the remaining ginsenoside compound K powder was not observed.

### Experimental Example 4

The constitution and the composition ratio of a water-solubilized mixture of ginsenoside compound K at a concentration of 40% (based on the content of ginsenoside compound K powder) (Sample 4), which is manufactured using ginsenoside compound K powder having a purity of 32.77%, are described in Table 5.

TABLE 6

| Constitution | Composition ratio (wt %) | Function (role) |
|---|---|---|
| Ginsenoside compound | 40.00 | Bioactive substance |
| K powder | | |
| 1,2-hexanediol | 30.00 | Solvent |
| Purified water | 16.00 | Solvent |
| 1,3-butanediol | 10.00 | Solution stabilizer |
| Ethanol | 4.00 | Dispersion agent |
| Total | 100.00 | |

The water-solubilized mixture of ginsenoside compound K (Sample 4) that was manufactured at the above-described composition ratio had a clear yellowish brown liquid phase, and the appearance of the remaining ginsenoside compound K powder was not observed.

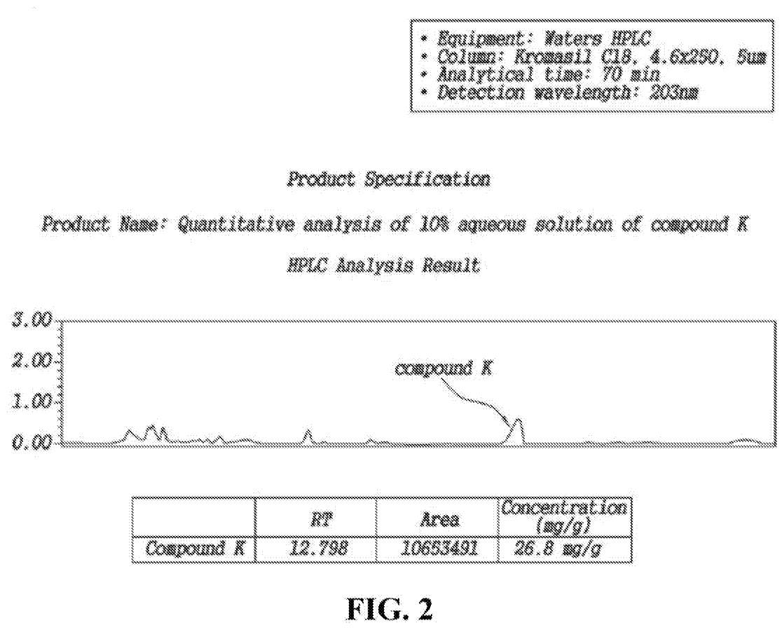

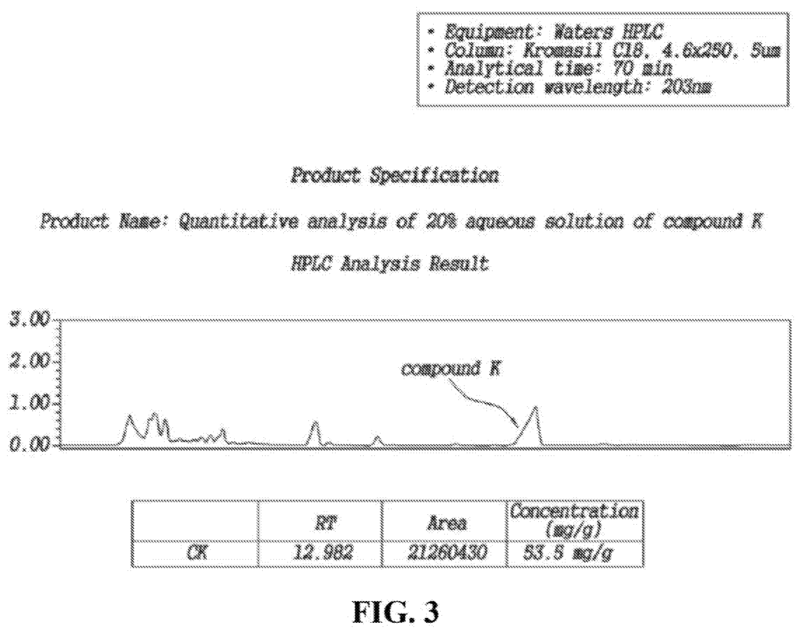

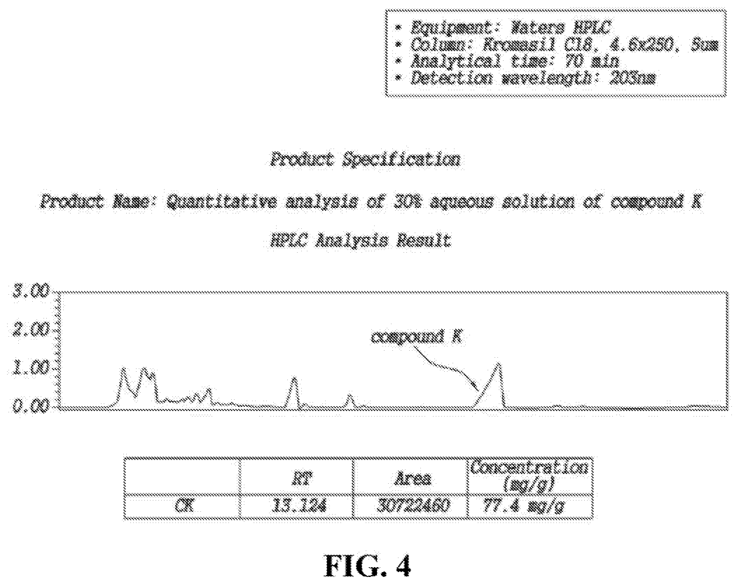

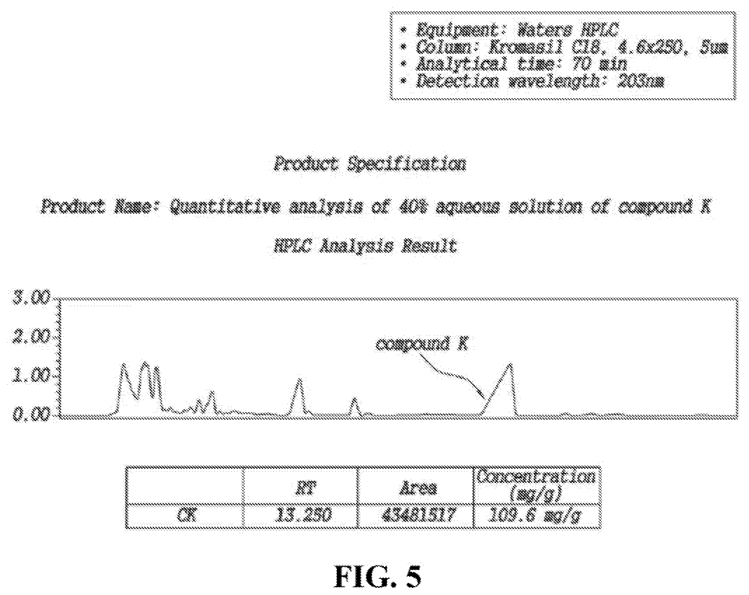

The concentrations of the water-solubilized mixture of ginsenoside compound K according to Experimental Example 1 (Sample 1), Experimental Example 2 (Sample 2), Experimental Example 3 (Sample 3), and Experimental Example 4 (Sample 4) were measured using high-performance liquid chromatography (HPLC). FIG. 2 is a view showing HPLC quantitative analysis results of an aqueous solution of ginsenoside compound K powder (purity 32.77%) at a concentration of 10%, and FIG. 3 is a view showing HPLC quantitative analysis results of an aqueous solution of ginsenoside compound K powder (purity 32.77%) at a concentration of 20%. Further, FIG. 4 is a view showing HPLC quantitative analysis results of an aqueous solution of ginsenoside compound K powder (purity 32.77%) at a concentration of 30%, and FIG. 5 is a view showing HPLC quantitative analysis results of an aqueous solution of ginsenoside compound K powder (purity 32.77%) at a concentration of 40%.

The concentration of the water-solubilized mixture of ginsenoside compound K using high-performance liquid chromatography (HPLC) is measured according to the following procedure.

i) Preparation of Sample

The water-solubilized mixtures of ginsenoside compound K of Experimental

Example 1 (Sample 1), Experimental Example 2 (Sample 2), Experimental Example 3 (Sample 3), and Experimental Example 4 (Sample 4) were manufactured using ginsenoside compound K powder having a purity of 32.77% obtained by processing Korean white fine root ginseng.

ii) Measurement Condition

Analysis equipment: Waters HPLC

Column: Kromasil C18, 4.6×250, 5 um

Analysis time: 70 min.

Detection: Measurement of UV absorbance at 203 nm using a UV detector

Condition: Water and acetonitrile were used as a mobile phase, separation was performed using a gradient elution method, and a flow rate was maintained at 1 ml/1 min.

TABLE 7
Table 7 shows the analysis conditions of high-performance
liquid chromatography (HPLC).

| Time (min.) | Water | Acetonitrile |
|---|---|---|
| 0 | 25% | 75% |
| 10 | 25% | 75% |
| 15 | 0% | 100% |
| 20 | 0% | 100% |

iii) Analysis Results

The concentration of the pure ginsenoside compound K contained in the aqueous solution, which contained ginsenoside compound K powder having a purity of 32.77% at concentrations of 10% (Sample 1), 20% (Sample 2), 30% (Sample 3), and 40% (Sample 4), was measured by LC-MS analysis. As a result, the content of the pure ginsenoside compound K contained in the water-solubilized mixture of ginsenoside compound K according to Experimental Example 1 (Sample 1), Experimental Example 2 (Sample 2), Experimental Example 3 (Sample 3), and Experimental Example 4 (Sample 4) is described in the following Table 8.

TABLE 8

| | Theoretical | | |
|---|---|---|---|
| | concentration | Analysis | Analysis |
| | [mg/g] | concentration of | concentration/ |
| | (Theoretical | high-performance | theoretical |
| | compound K | liquid chromatography | concentration |
| Sample | concentration) | (HPLC) [mg/g] | [%] |
| Sample 1 (10% | 32.77 | 26.80 | 81.78 |
| concentration) | | | |
| Sample 2 (20% | 65.54 | 53.50 | 81.63 |
| concentration) | | | |
| Sample 3 (30% | 98.31 | 77.40 | 78.73 |
| concentration) | | | |
| Sample 4 (40% | 131.08 | 109.60 | 83.61 |
| concentration) | | | |

IV) Interpretation of Quantitative Analysis Results of the Water-Solubilized Mixture of the Ginsenoside Compound K

The theoretical concentration of the pure ginsenoside compound K contained in 10%, 20%, 30%, and 40% concentrations of the water-solubilized mixture of ginsenoside compound K was 32.77 mg/g, 65.54 mg/g, 98.31 mg/g, and 131.08 mg/g. However, the results of actual LC-MS analysis showed that the concentration of the pure ginsenoside compound K in 10%, 20%, 30%, and 40% concentrations of the water-solubilized mixture of ginsenoside compound K was actually measured to be 26.80 mg/g, 53.50 mg/g, 77.4 mg/g, and 109.60 mg/g, which were different from the theoretical concentrations.

The difference in the concentration of the water-solubilized mixture of ginsenoside compound K is a phenomenon occurring due to the reason that a bulking agent, which was inevitably used in a process of manufacturing ginsenoside compound K powder during water-solubilization of ginsenoside compound K and thus was contained in ginsenoside compound K powder, was removed and lost during water-solubilization of the ginsenoside compound K.

The water-solubilized mixture of ginsenoside compound K which is the present invention may be used as a composition for functional cosmetics. A variety of constitutional components that can be used as permitted by law and according to the purposes may be added to the composition for functional cosmetics. The constitutional components may be added according to specific purposes such as the composition for functional cosmetics applied to skin whitening and the composition for functional cosmetics applied to anti-aging.

The product may be any type obtained using water as a solvent. Specifically, the product may be a liquid-type product such as an aqueous solution or a suspension, and may also be a semisolid-type product such as a paste, gel, cream, and lotion.

The composition for functional cosmetics, which is the present invention, may further include at least one additive selected from the group consisting of a flavoring agent, a pigment, a stabilizer, a vitamin, a carrier, a bactericide, an antioxidant, a preservative, a moisturizer, a thickener, an inorganic salt, a synthetic polymer substance, oil, water, a surfactant, alcohol, and a chelating agent, thus being used to manufacture the functional cosmetics.

Specifically, when the composition for functional cosmetics, which is the present invention, is manufactured as a liquid cosmetic material composition such as a solution and a suspension, the composition may be manufactured so as to further include ethanol, isopropanol, ethyl carbonate, ethyl acetate, benzyl alcohol, benzyl benzoate, propanediol, 1,3-butanediol oil, glycerol aliphatic ester, polyethylene glycol, fatty acid ester of sorbitan, microcrystalline cellulose, aluminum metahydroxide, bentonite, or agar as a carrier component, in addition to water.

When the composition for functional cosmetics which is the present invention is manufactured as the semisolid-type cosmetic material composition such as paste, cream, lotion, or gel, the composition may be manufactured so as to further include starch, tragacanth, cellulose derivatives, polyethylene glycol, silicone, bentonite, silica, talc, or zinc oxide as a carrier component, in addition to water.

The composition for functional cosmetics which is the present invention may be manufactured so as to include skin-whitening components, skin-wrinkle reduction components, or ultraviolet-ray-blocking components known in the art in order to enhance or add skin-related activity. Specific examples of such components may be found in Korean Functional Cosmetic Standards Codex according to the Cosmetic Act. Examples of skin-whitening components may include arbutin, niacinamide, ascorbyl glucoside, alpha-bisabolol, and oil-soluble licorice (glycyrrhiza) extracts.

The functional cosmetics which are the present invention may be used for various purposes such as a cosmetic essence, tonic, cosmetic cream, cosmetic lotion, cosmetic beauty pack, cosmetic mist, cosmetic ampoule, cosmetic skin, nutritional cream, massage cream, cleansing cream, cleansing foam, cleansing water, cleansing oil, milky lotion, soap, liquid cleanser, bathing agent, sunscreen cream, sun oil, shampoo, rinse, hair treatment, hair mousse, hair liquid, pomade, hair-coloring agent, hair-bleaching agent, color rinse, hair tonic, or scalp treatment.

### Application Example 1

The composition of cosmetic skin is shown in Table 9, which is manufactured by a typical method using a water-solubilized mixture of ginsenoside compound K at a concentration of 30% (based on the content of ginsenoside compound K powder) (Sample 3) manufactured using ginsenoside compound K powder having a purity of 32.77%.

TABLE 9

| Phase | Raw material name | Content (%) | Note |
|---|---|---|---|
| A | Purified water | To 100.00 | |
| phase | EDTA-2Na | 0.05 | |
| | Allantoin | 0.1 | |
| | Glycerin | 10.0 | |
| | Natural Preservative | 2.0 | |
| | Xanthan Gum | 0.08 | |
| B | Water-solubilized mixture of ginsenoside | 10.0 | |
| phase | compound K at a concentration of 30% | | |
| C | Dipropylene Glycol | 4.0 | |
| phase | Tween 20 | 0.5 | |
| | Fragrance | QS | |

Work procedure: 1) An A phase is dissolved. 2) A B phase is slowly added to the A phase while agitating. 3) While the A+B phase is agitated, a C phase is added. 4) Filtration is performed using a 200-mesh filter, followed by aging in a reservoir.

### Application Example 2

The composition of cosmetic cream is shown in Table 10, which is manufactured by a typical method using a water-solubilized mixture of ginsenoside compound K at a concentration of 30% (based on the content of ginsenoside compound K powder) (Sample 3) manufactured using ginsenoside compound K powder having a purity of 32.77%.

TABLE 10

| Phase | Raw material name | Content (%) | Note |
|---|---|---|---|
| A | Purified water | To 100.00 | |
| phase | Glycerin | 10.00 | |
| | EDTA-2Na | 0.05 | |
| | Allantoin | 0.20 | |
| | L-Arginine | 0.12 | |
| | Xanthan Gum | 0.08 | |
| | Water-solubilized mixture of ginsenoside | 10.00 | |
| | compound K at a concentration of 30% | | |
| B | Cetearyl Alcohol | 2.00 | |
| phase | Stearic Acid | 1.00 | |
| | Sunflower Oil | 6.00 | |
| | Jojoba Oil | 4.00 | |
| | Vit E Acetate | 0.20 | |
| | Sorbitan Sesquioleate | 1.00 | |
| | Cetearyl Glucoside/Cetearyl Alcohol | 2.00 | |
| C | Carbomer | 0.12 | |
| phase | | | |
| D | Preservative | QS | |
| phase | | | |

### Application Example 3

The composition of cosmetic lotion is shown in Table 11, which is manufactured by a typical method using a water-solubilized mixture of ginsenoside compound K at a concentration of 10% (based on the content of ginsenoside compound K powder) (Sample 1) manufactured using ginsenoside compound K powder having a purity of 32.77%.

TABLE 11

| Phase | Raw material name | Content (%) | Note |
|---|---|---|---|
| A | Purified water | To 100 | |
| phase | EDTA-2Na | 0.04 | |
| | Allantoin | 0.10 | |
| | Xanthan Gum | 0.06 | |
| | L-Arginine | 0.10 | |
| | Na-Hyaluronate (1%) | 2.00 | |
| | Glycerin | 5.00 | |
| | Water-solubilized mixture of ginsenoside | 5.00 | |
| | compound K at a concentration of 10% | | |
| B | Cetearyl Alcohol | 1.00 | |
| phase | Stearic Acid | 0.50 | |
| | Bees Wax | 0.40 | |
| | Sunflower Oil | 4.00 | |
| | Macadamia Oil | 3.00 | |
| | Vit E Acetate | 0.20 | |
| | Dimethicone | 0.50 | |
| C | Carbopol 940 | 0.08 | |
| phase | | | |
| D | Preservative | QS | |
| phase | | | |
| E | Camellia sinensis Leaf Extract | 1.00 | |
| phase | Beta-Glucan | 1.00 | |

### Application Example 4

The composition of cosmetic ampoule is shown in Table 12, which is manufactured by a typical method using a water-solubilized mixture of ginsenoside compound K at a concentration of 40% (based on the content of ginsenoside compound K powder) (Sample 4) manufactured using ginsenoside compound K powder having a purity of 32.77%.

TABLE 12

| Phase | Raw material name | Content (%) | Note |
|---|---|---|---|
| A | Purified water | To 100 | |
| phase | Glycerin | 10.00 | |
| | EDTA-2Na | 0.04 | |
| | Xanthan Gum | 0.10 | |
| | Na-Hyaluronate (1%) | 3.00 | |
| | Allantoin | 0.10 | |
| | Beta-Glucan | 4.00 | |
| | Water-solubilized mixture of ginsenoside | 8.00 | |
| | compound K at a concentration of 40% | | |
| | Preservative | QS | |
| B | Dipropylene Glycol | 5.00 | |
| phase | HCO 60 | QS | |
| | Fragrance | 0.02 | |
| C | Camellia sinensis Leaf Water | 2.00 | |
| phase | Portulaca oleracea Extract | 1.00 | |
| | Hydrolyzed Collagen | 2.00 | |
| | Centella asiatica Extract | 2.00 | |
| | Hippophae rhamnoides Extract | 1.00 | |

### Application Example 5

The composition of cosmetic mist is shown in Table 13, which is manufactured by a typical method using a water-solubilized mixture of ginsenoside compound K at a concentration of 10% (based on the content of ginsenoside compound K powder) (Sample 1) manufactured using ginsenoside compound K powder having a purity of 32.77%.

TABLE 13

| Phase | Raw material name | Content (%) | Note |
|---|---|---|---|
| A | Purified water | To 100 | |
| phase | EDTA-2Na | QS | |
| | Allantoin | 0.10 | |
| | Hydrolyzed Collagen | 2.00 | |
| | 1,3-Butylene Glycol | 3.00 | |
| | Sodium PCA | 2.00 | |
| | Water-solubilized mixture of ginsenoside | 3.00 | |
| | compound K at a concentration of 10% | | |
| B | Dipropylene Glycol | 4.00 | |
| phase | Tween 20 | 0.50 | |
| | Fragrance | 0.08 | |
| C | Centella asiatica Extract | 1.50 | |
| phase | | | |

### Application Example 6

The composition of cosmetic essence is shown in Table 14, which is manufactured by a typical method using a water-solubilized mixture of ginsenoside compound K at a concentration of 20% (based on the content of ginsenoside compound K powder) (Sample 2) manufactured using ginsenoside compound K powder having a purity of 32.77%.

TABLE 14

| Phase | Raw material name | Content (%) | Note |
|---|---|---|---|
| A | Purified water | To 100 | |
| phase | Glycerin | 10.00 | |
| | EDTA-2Na | 0.04 | |
| | Allantoin | 0.10 | |
| | Xanthan Gum | 0.06 | |
| | L-Arginine | 0.12 | |
| | Beta-Glucan | 3.00 | |
| | Water-solubilized mixture of ginsenoside | 10.00 | |
| | compound K at a concentration of 20% | | |
| B | Cetearyl Alcohol | 1.50 | |
| phase | Stearic Acid | 0.50 | |
| | Bees Wax | 0.30 | |
| | Jojoba Oil | 3.00 | |
| | Safflower Oil | 5.00 | |
| | Vitamin E Acetate | 0.30 | |
| | Dimethicone | 0.50 | |
| C | Carbopol 940 | 0.10 | |
| phase | | | |
| D | Fragrance | 0.08 | |
| phase | | | |
| E | Na-Hyaluronate (1%) | 2.00 | |
| phase | Sodium PCA | 2.00 | |

### Application Example 7

The composition of a cosmetic beauty pack is shown in Table 15, which is manufactured by a typical method using a water-solubilized mixture of ginsenoside compound K at a concentration of 20% (based on the content of ginsenoside compound K powder) (Sample 2) manufactured using ginsenoside compound K powder having a purity of 32.77%.

TABLE 15

| Phase | Raw material name | Content (%) | Note |
|---|---|---|---|
| A | Purified water | To 100.00 | |
| phase | Polyvinyl Alcohol | 15.00 | |
| | Sodium Carboxymethyl Cellulose | 0.25 | |
| | Glycerin | 4.00 | |
| | Allantoin | 0.15 | |
| B | Ethanol | 5.00 | |
| phase | PEG-Nonylphenyl Ether | 0.30 | |
| | Polysorbate 60 | 0.30 | |
| | Preservative | QS | |
| | Fragrance | QS | |
| C | Water-solubilized mixture of ginsenoside | 2.00 | |
| phase | compound K at a concentration of 20% | | |

While the present invention has been described with reference to exemplary embodiments thereof, it is to be understood that the present invention is not limited to the disclosed exemplary embodiments, but on the contrary, those skilled in the art will appreciate that various amendments and modifications are possible from the description. Accordingly, it is intended that the idea of the present invention be defined only by the claims appended hereto, and that all equivalents or equivalent variations thereof fall within the scope of the present invention.

1. A water-solubilized mixture of ginsenoside compound K, comprising:
hexanediol;
polyol;
ginsenoside compound K; and
water.

2. The water-solubilized mixture of ginsenoside compound K of claim 1, wherein the hexanediol is 1,2-hexanediol.

3. The water-solubilized mixture of ginsenoside compound K of claim 2, wherein the polyol is 1,3-butanediol.

4. The water-solubilized mixture of ginsenoside compound K of claim 3, wherein a content of ginsenoside compound K is 0.01 to 40.00 wt %, a content of the 1,2-hexanediol is 0.01 to 35.00 wt %, a content of the 1,3-butanediol is 0.01 to 30.00 wt %, and a content of the water is 0.01 to 40.00 wt %.

5. The water-solubilized mixture of ginsenoside compound K of claim 4, further comprising:
0.01 to 4.00 wt % of ethanol.

6. A composition for functional cosmetics, comprising:
the water-solubilized mixture of ginsenoside compound K of claim 1.

7. The composition for functional cosmetics of claim 6, further comprising:
at least one additive selected from the group consisting of a flavoring agent, a pigment, a stabilizer, a vitamin, a carrier, a bactericide, an antioxidant, a preservative, a moisturizer, a thickener, an inorganic salt, a synthetic polymer substance, oil, water, a surfactant, alcohol, and a chelating agent.

8. The composition for functional cosmetics of claim 7,
wherein the composition for functional cosmetics is cosmetic essence, tonic, cosmetic cream, cosmetic lotion, cosmetic beauty pack, cosmetic mist, cosmetic ampoule, cosmetic skin, nutritional cream, massage cream, cleansing cream, cleansing foam, cleansing water, cleansing oil, milky lotion, soap, liquid cleanser, bathing agent, sunscreen cream, sun oil, shampoo, rinse, hair treatment, hair mousse, hair liquid, pomade, hair-coloring agent, hair-bleaching agent, color rinse, hair tonic, or scalp treatment.

9. A method of manufacturing a water-solubilizing ginsenoside compound K, the method comprising:
(1) manufacturing a first mixture of ginsenoside compound K powder and hexanediol by adding hexanediol to ginsenoside compound K powder and mixing the resultant;
(2) manufacturing a second mixture of ginsenoside compound K powder, the hexanediol, and polyol by adding polyol to the first mixture and mixing the resultant; and
(3) manufacturing an aqueous solution containing ginsenoside compound K by adding the second mixture to water at 65 to 75° C. during agitation and performing mixing.

10. The method of claim 9, wherein a content of the ginsenoside compound K is 0.01 to 40.00 wt %, a content of the hexanediol is 0.01 to 35.00 wt %, a content of the polyol is 0.01 to 30.00 wt %, and a content of the water is 0.01 to 40.00 wt %.

11. The method of claim 10, wherein the polyol is any selected from the group consisting of butanediol, dipropylene glycol, glycerin, and propanediol.

12. The method of claim 11, further comprising:
before manufacturing the first mixture, in order to reduce a water-solubilization time of the ginsenoside compound K,
a step of manufacturing a dispersion substance of ginsenoside compound K powder by mixing the ginsenoside compound K powder with ethanol and performing dispersion, and
a step of adding the hexanediol to the dispersion substance of ginsenoside compound K powder and mixing the resultant, thus manufacturing the first mixture.

13. The method of claim 12, wherein a content of the ethanol is 0.01 to 4.00 wt %.

14. The method of claim 13,
wherein the manufacturing of the first mixture performed by adding 1,2-hexanediol at 55 to 65° C. to the dispersion substance of ginsenoside compound K powder and mixing the resultant, and
wherein the manufacturing of the second mixture performed by adding 1,3-butanediol at 55 to 65° C. to the first mixture and mixing the resultant.

15. The method of claim 14, further comprising:
after the manufacturing of the aqueous solution containing the ginsenoside compound K,
manufacturing an aged aqueous solution containing ginsenoside compound K by cooling the aqueous solution containing ginsenoside compound K to 30 to 40° C., followed by aging the aqueous solution containing ginsenoside compound K at room temperature for 2 to 4 days; and
manufacturing a purified aqueous solution containing ginsenoside compound K by filtering the aged aqueous solution containing ginsenoside compound K.
